(19) United States
(12) Patent Application Publication
(10) Pub. No.: US 2023/0247983 A1
(43) Pub. Date: Aug. 10, 2023

(54) Cryogenic Storage Container

(71) Applicant: SEXTON BIOTECHNOLOGIES, INC., Indianapolis, IN (US)

(72) Inventors: Adam Shields, Noblesville, IN (US); Frank J. Fischer, JR., Bloomington, IN (US); Michael Pallotta, Carmel, IN (US); Sean Werner, Indianapolis, IN (US)

(21) Appl. No.: 18/165,792

(22) Filed: Feb. 7, 2023

Related U.S. Application Data
(60) Provisional application No. 63/307,614, filed on Feb. 7, 2022.

(51) Int. Cl. A01N 1/02

(52) U.S. Cl. CPC A01N 1/0263

(57) ABSTRACT

Containers for cryopreservation of biological material are disclosed. Each container is configured to provide a substantially consistent freeze profile throughout a chamber of the container. Specific embodiments include containers having a uniform dimension between two opposing walls defining the chamber, and containers wherein a ratio of a sum of the interior surface areas to the volume of the chamber is at or between 1 to 5 and 2 to 5.

### REFERENCE TO RELATED APPLICATIONS

This application claims one or more inventions which were disclosed in U.S. Provisional Patent Application No. 63/307,614, filed Feb. 7, 2022, titled “Cryogenic Storage Container”. The benefit under 35 USC § 119(e) of this United States provisional application is hereby claimed, and the aforementioned application is hereby incorporated herein by reference.

### BACKGROUND OF THE INVENTION

The present invention generally pertains to devices for cryopreservation of cells and/or tissues, and more specifically to cryogenic storage containers (i.e., “cryostorage containers”) useful for the cryopreservation of biological material (e.g., cells and/or tissues), such as mammalian cells and tissue samples/specimens.

Cells and tissues are frequently cryopreserved to temporally extend their viability and usefulness in biomedical applications. The process of cryopreservation involves, in part, placing cells into aqueous solutions containing electrolytes and chemical compounds that protect the cells during the freezing process (cryoprotectants). The freezing process, however, is not as benign as one might assume; it generally induces extreme variations in chemical, thermal, and electrical properties that could be expected to alter intracellular organelles, cellular membranes, and the delicate cell-cell interaction systems associated with tissues and organs.

As aqueous solutions containing cryoprotectants and cells are cooled to temperatures slightly below their freezing point, the solutions remain in the liquid state. This condition in which such a solution remains liquid below its phase transition temperature is termed supercooling. As the aqueous solutions are cooled further below their freezing point, the extent of supercooling increases. In the absence of intervention, the water molecules in the solution will, at a point usually no more than 15° C. below the freezing point, spontaneously crystallize, and pure water will precipitate as ice.

During the transition from the liquid to the solid state, the solution moves from a higher to a lower free energy state, resulting in an exothermic reaction. The heat produced during this phase transition causes a transient warming of the sample during which the sample temperature increases. Meanwhile the surrounding environment (e.g., the device in which the sample is being cryopreserved) either remains at a constant temperature or continues to cool (depending upon the cooling approach used).

Subsequently, as the heat in the sample dissipates, the thermal disequilibrium between the sample and cooling device created during this event causes the sample to undergo a rapid cooling rate to re-establish thermal equilibrium. In many cases this rapid cooling rate causes the formation of intracellular ice, which usually results in cell death. This formation of intracellular ice is typically dependent upon the mass of the sample, the heat transfer properties of the sample container, the cooling protocol used, and the fundamental cryobiological properties of the cells.

Notably, deviations from the cooling rate and/or thawing rate prescribed for a particular cell type can lead to cell damage and/or cell death during the process. Conventional cryogenic storage containers useful to directly hold biological material for cryopreservation have shapes and sizes that enable or result in deviations of the cooling rate and/or thawing rate across a volume of the biological material. Vials, for example, which are conventionally used to directly hold biological material during cryopreservation, can yield significant variations in the cooling rate and/or thawing rate across a volume of biological material contained therein. Vials are often positioned within a cryostorage/freezer box, which holds multiple samples and aids in the locating and retrieval of the vials from cryostorage. Some boxes aid in limiting the freezing rate of the biological sample positioned within the vial. For example, some cryostorage/freezer boxes are formed of a foam material that provides thermal insulation. The freezing profile of the sample within the vial, however, is still impacted by the shape and size of the vial.

In small cylindrical vials (e.g., large enough to hold 5 ml or less) substantially all the sample freezes at the same rate. In larger diameter cylindrical vials (e.g., large enough to hold 10 ml or more, 20 ml or more, 30 ml or more, 40 ml or more, and/or 50 ml or more), however, portions in the middle of these larger diameter vials take substantially longer to freeze. These middle portions, therefore, have different cell viability results than other portions within these larger diameter vials.

There remains a need for devices that reduce and/or avoid the problems associated with the disequilibrium conditions described above. Thus, there is a need for improvement in this field.

### SUMMARY OF THE INVENTION

Containers for cryopreservation, as disclosed herein, are arranged to provide a substantially consistent freeze profile throughout a chamber of the container. Such arrangements facilitate freezing of the contents of the cryostorage container, such as cells, at a constant rate or a more constant rate to reduce the likelihood of damage to the cells during freeze and/or thaw cycles. (In some instances herein, the terms “freeze cycle”, “freeze profile”, or another similar term are used for brevity. It should be understood, however, that containers according to the present disclosure may provide similar advantages to thawing cycles, thawing profiles, and the like).

Cryopreservation containers disclosed herein can be provided with uniform characteristic dimensions and high dimensional aspect ratios while facilitating or maintaining freeze/thaw consistency and while having relatively large capacities compared to conventional or previously existing vials and bags for cryostorage. For example, the cryopreservation containers disclosed herein can be arranged to hold 30 ml or more.

Some embodiments of a cryostorage container may comprise a serpentine chamber for holding a volume of liquid, a vent opening and a drain opening each communicating with the serpentine chamber, a filter associated with the vent opening to filter air entering and/or exiting the serpentine chamber when in communication with a surrounding environment, a first end region, and a second end region. The serpentine chamber may be defined at least partially by a first wall and a second wall, wherein the first wall follows a serpentine path from the first end region of the cryostorage container to the second end region of the cryostorage container. The serpentine path can be defined by a series of straight portions forming a zig-zag or chevron pattern. The serpentine path can additionally or alternatively be defined at least partially by a series of curves bending in alternating directions. The series of curves can be separated by straight portions.

In some embodiments, the second wall can be spaced from the first wall along the serpentine path such that the chamber has a uniform width between the first wall and the second wall along the serpentine path. For example, the width of the chamber between the second wall and the first wall, measured along a vector normal to the first wall, can be at or between 5 mm and 7 mm.

In some embodiments, the cryostorage container can comprise heat-sealable tubing attached to the drain opening and/or the vent opening. The heat-sealable tubing can retain a filter, particularly if the heat-sealable tubing is attached to the vent opening.

In some embodiments, the vent opening can be positioned at a first end of the serpentine chamber and/or the drain opening can be positioned at a second end of the serpentine chamber. The serpentine path can have a length extending from the first end to the second end. The length can be greater than a width and/or a height of the serpentine chamber.

In some embodiments, the cryostorage container can comprise a hanger opening arranged to receive an IV bag hanger. The hanger opening can be positioned at an opposing end of the cryostorage container relative to the drain opening such that when the cryostorage container hangs freely from an IV hanger, the drain opening is at the lowest portion of the serpentine chamber. The hanger opening can be off-center along the end of the cryostorage container.

Cryostorage containers of the present disclosure may comprise: a chamber having a volume; the chamber defined by wall portions of the cryostorage container and wherein the wall portions of the cryostorage container each have an interior surface facing the chamber and an exterior surface opposing the interior surface; and wherein each interior surface has a surface area and wherein a ratio of a sum of the surface areas to the volume of the chamber is at or between 1 to 5 and 2 to 5. The wall portions defining the chamber may follow a serpentine path from a first end of the chamber to a second end of the chamber. The chamber may have a length extending from the first end to the second end and a uniform width along the length. The width may be at or between 5 mm and 7 mm. The ratio may be at or between 3 to 10 and 2 to 5. In any of the disclosed arrangements, the ratio can be about 1 to 3.

The wall portions of the containers may have a thermal conductivity at or between 0.16×10-3 watts per Kelvin and 0.32×10-3 watts per Kelvin at room temperature. In any of the disclosed arrangements, the wall portions may comprise a material having a thermal conductivity at or between 0.10 to 0.20 W/m K at room temperature. The body and/or lid may be formed of a polymer such as cyclic olefin copolymer. The polymer may be doped with nanoparticles (e.g., metals) to increase the thermal conductivity of the material.

Advantageously, the exemplary cryostorage container illustrated in the provided figures can be oriented in multiple directions. For example, the cryostorage container can be positioned with the feet on a supporting surface and the vent and drain openings of the lid located at an uppermost portion of the cryostorage container. The cryostorage container may also be oriented with the drain opening positioned at the lowest point of the chamber so that essentially all the liquid contained in the chamber will drain out through the drain opening. In such an arrangement, the vent opening will also be positioned at an upper portion of the chamber so as to allow air to enter the chamber without mixing in the liquid being drained and/or allowing liquid to escape the chamber through the vent opening. During a drain procedure, air entering the chamber through the vent opening is filtered by the filter associated with the vent opening to avoid contamination of the interior volume of the serpentine chamber.

It will be appreciated by those of skill in the art that the cryostorage container may be formed by a variety of manufacturing techniques, such as injection molding, blow molding, rotational molding, gas assist molding, and rapid prototyping, just to name a few non-limiting examples. The body and lid may be manufactured separately and then attached together, such as by adhesive, ultrasonic welding, hot plate welding, infrared welding, and/or laser welding. In some of the manufacturing techniques (e.g., rapid prototyping) the lid and body may be manufactured as a unitary body. The cryostorage container in the illustrated embodiments can be formed by at least injection molding and rapid prototyping. Preferably, regardless of manufacturing technique, the container will be hermetically sealable.

Cryostorage containers of this disclosure preferably have rigid walls that maintain the same shape when filled or drained, and regardless of the volume of the stored contents. Such arrangements can also provide a more consistent ratio of surface area to volume for the fluid in the container than bags or other flexible storage containers. Advantageously, such arrangements can provide greater predictability and reliability during freezing and thawing cycles, which can help promote cell viability.

Additionally, rigid containers can be less prone to leakage than bags and, therefore, may be more preferred for situations when the contents being stored are toxic or hazardous. One or more inner surfaces of the walls of the container (e.g., the inner surfaces of the first and second walls), may be hydrophobic. Advantageously, such an arrangement can reduce cell adhesion.

A nucleation site for ice crystal formation may be included on an inner surface of a wall defining the chamber. For example, the inner surface may include a coating with an ice nucleating agent (INA). Such coatings may be protected by a water permeable top layer (e.g., titanium carbide, molybdenum disulfide with tungsten chalcogenides, and/or boron nitride with graphyne). By “seeding” the ice formation, random formation of ice in the liquid during freezing can be reduced and/or eliminated, thus increasing sample survival rates. Random ice formation can occur at unpredictable temperatures; consequently, sample survival rates can be highly variable between repeated trials with the same freezing protocol. Furthermore, the extremely rapid crystallization which results when ice forms in a highly supercooled solution can cause damage to cells and tissues. A nucleation site provides control and increases consistency of freezing profile across samples and batches.

Advantageously, the arrangements disclosed herein can provide a cryostorage container having a chamber with a constant surface area per volume ratio along the length, width, and/or height of the chamber. For example, the chamber may have a volume defined by wall portions of the cryostorage container and wherein the wall portions of the cryostorage container each have an interior surface facing the chamber and an exterior surface opposing the interior surface and wherein each interior surface has a surface area and

wherein a ratio of the sum of the surface areas to the volume of the chamber is at or between 1 to 5 and 2 to 5.

Containers of the present disclosure can also exhibit greater compressive strength than linear containers having the same wall thickness. Advantageously, the serpentine path of the side walls of the container increase the compressive strength of the structure in at least one direction relative to a linear-wall container.

Walls of the containers may include one or more thermocouples. The thermocouple(s) may be embedded in the wall. In some embodiments, one or more walls of the containers may include one or more mounting features to facilitate consistent and reliable coupling of thermocouples to the containers. The thermocouple(s) may be separated from the sample so as to not disturb nucleation behavior.

Containers may include electrodes arranged to apply high electric fields, in the kilovolt range, for electrofreezing and/or electroporation purposes. Such electrodes may be coupled to one or more walls of the container.

Containers may include a displacement body that protrudes into the chamber of the container (e.g., the serpentine chamber). For example, one or more fins or rods may extend into the chamber and displace a volume of sample therein. Advantageously, such arrangements may further reduce the distance between a portion of the sample and a surface of the container. The displacement body/bodies may be arranged to remove heat from a sample. For example, the displacement body/bodies may have thermal conductivity greater than that of walls of the container. As one particular example, the displacement body/bodies may comprise a metal and the container walls may comprise plastic. A thermocouple may also extend into the sample volume defined by the container walls.

Containers may include identifications tags. Such tags may include electronics for the remote detection of the container and/or transfer of information therefrom (e.g., RFID, Bluetooth, etc.). Containers may include graduation marks indicative of the volume of a sample in the chamber when the container is in a particular orientation.

Containers may be optically clear, or otherwise include structures, characteristics, and/or materials, to facilitate visual inspection, optical quantitative measurement, and other types of non-destructive testing of the stored contents (for example, spectroscopy, counting, fluorescence, and the like). In some embodiments, containers include cuvette like optical characteristics.

Methods of using the containers disclosed herein include removing of oxygen from the chamber and/or purging the chamber with nitrogen gas through one or more openings. Electrical, mechanical, optical, and/or acoustical measuring of liquid level through one or more openings. Introducing INAs, sensors (e.g., optical and/or electrical such as a thermocouple), cryoprotectants, and/or cells into the chamber through one or more openings. Placing the container in a cryogenic freezer and/or placing the container in a cryopreserved cell thawer, such as the VIA Thaw CB1000 by GE Lifesciences.

The container disclosed herein may be hermetically sealed with cells and/or cryoprotectant positioned in the chamber. The containers disclosed herein may be provided in a sealed, sterile package.

### BRIEF DESCRIPTION OF THE DRAWINGS

FIG. 1 is a perspective view of a cryostorage container according to an embodiment of the present invention.

FIG. 2 is a perspective view of a body of the cryostorage container of FIG. 1.

FIG. 3 is a side view of the cryostorage container of FIG. 1 in a suspended/draining configuration.

FIG. 4 is a side view of the cryostorage container of FIG. 1 in a filling configuration.

FIG. 5 is a side view of the cryostorage container of FIG. 1 in a draining configuration.

FIGS. 6, 7, and 8 are perspective views of the body of the cryostorage container of FIG. 1.

FIGS. 9 and 10 are cross-sectional views of the body of the cryostorage container of FIG. 1.

FIGS. 11 and 12 are perspective views of a lid of the cryostorage container of FIG. 1.

FIG. 13 is a perspective view of a chamber for a cryostorage container according to another embodiment of the present invention.

FIG. 14 is a perspective view of a chamber for a cryostorage container according to a further embodiment of the present invention.

FIG. 15 is a cross-sectional view of a chamber for a cryostorage container according to yet another embodiment of the present invention.

FIG. 16 is a perspective view of a chamber for a cryostorage container according to a further embodiment of the present invention.

FIG. 17 is a partial cross-sectional view of the chamber of FIG. 16.

FIG. 18 is a cross-sectional view of a chamber for a cryostorage container according to yet another embodiment of the present invention.

FIG. 19 is a cross-sectional view of a chamber for a cryostorage container according to a further embodiment of the present invention.

FIG. 20 is a cross-sectional view of a chamber for a cryostorage container according to yet another embodiment of the present invention.

FIG. 21 is a cross-sectional view of a chamber for a cryostorage container according to a further embodiment of the present invention.

FIG. 22 is a detail cross-sectional view of the chamber of FIG. 21.

FIG. 23 is a cross-sectional view of a chamber for a cryostorage container according to yet another embodiment of the present invention.

FIG. 24 is a perspective view of a chamber for a cryostorage container according to a further embodiment of the present invention.

FIG. 25 is a perspective view of a chamber for a cryostorage container according to yet another embodiment of the present invention.

FIG. 26 is a partial cross-sectional view of the chamber of FIG. 25.

FIG. 27 is a partial perspective view of a cryostorage container according to yet another embodiment of the present invention.

FIG. 28 is a schematic partial cross-sectional view of the cryostorage container of FIG. 27 and a nucleation trigger.

FIG. 29 is another schematic partial cross-sectional view of the cryostorage container and the nucleation trigger of FIG. 28.

FIG. 30 is a partial perspective view of a cryostorage container according to yet another embodiment of the present invention.

FIG. 31 is a partial perspective view of the cryostorage container of FIG. 30 and a thermocouple.

FIG. 32 is a perspective cross-sectional view of a cryostorage container according to yet another embodiment of the present invention.

FIG. 33 shows a perspective view of an internal side of a lower wall of a cryostorage container according to an embodiment of the present invention.

FIG. 34 shows a perspective view of an external side of an upper wall of the cryostorage container of FIG. 33.

FIG. 35 shows a front view of an internal side of a lower wall of a cryostorage container according to an embodiment of the invention.

FIG. 36 shows a planar cross section of the cryostorage container of FIG. 35.

### DETAILED DESCRIPTION OF THE INVENTION

For the purpose of promoting an understanding of the principles of the invention, reference will now be made to the embodiments illustrated in the drawings and specific language will be used to describe the same. It should nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

The language used in the claims and the written description is to only have its plain and ordinary meaning, except for terms explicitly defined below. Such plain and ordinary meaning is defined here as inclusive of all consistent dictionary definitions from the most recently published (on the filing date of this document) general purpose Merriam-Webster dictionary.

The terms “fill”, “filling”, and “filled” as used herein include both partial and complete filling. The term “drain”, “draining”, and “drained” as used herein include both partial and complete draining. The term “vent” as used herein means an opening to allow equalization of gas pressure between the inside and outside of the cryostorage container. Vents may allow the passage of gas through the vent (e.g., through a filter) and/or include a flexible membrane (e.g., a bag and/or balloon) that allows for expansion and/or contraction of gas within the chamber of the cryostorage container. The term “chamber width” as used herein means the distance between inside surfaces on opposing sides of the chamber, measured along a vector normal (i.e., perpendicular) to at least one of the inside surfaces. The “tubes”, “tubing” and “flexible tubing” disclosed herein can comprise a material suitable for cryostorage at −196° C. or below. Further forms, objects, features, aspects, benefits, advantages, and embodiments of the present invention will become apparent from a detailed description and drawings provided herewith

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

The terms “approximately” and “about”, when qualifying a quantity, size, or geometry, shall mean the given value with a tolerance plus or minus 10 percent of the value, unless otherwise specified.

All ranges disclosed herein, unless otherwise indicated, encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

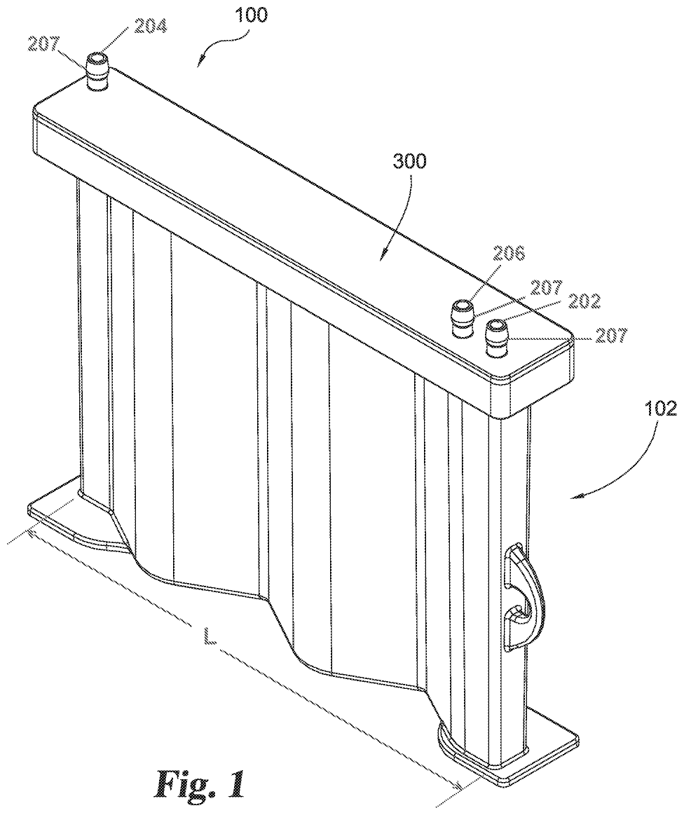

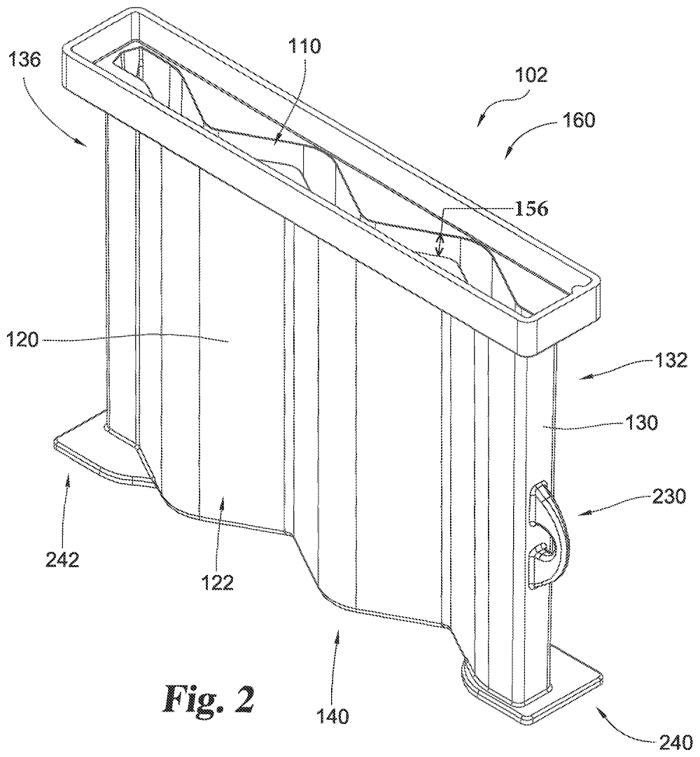

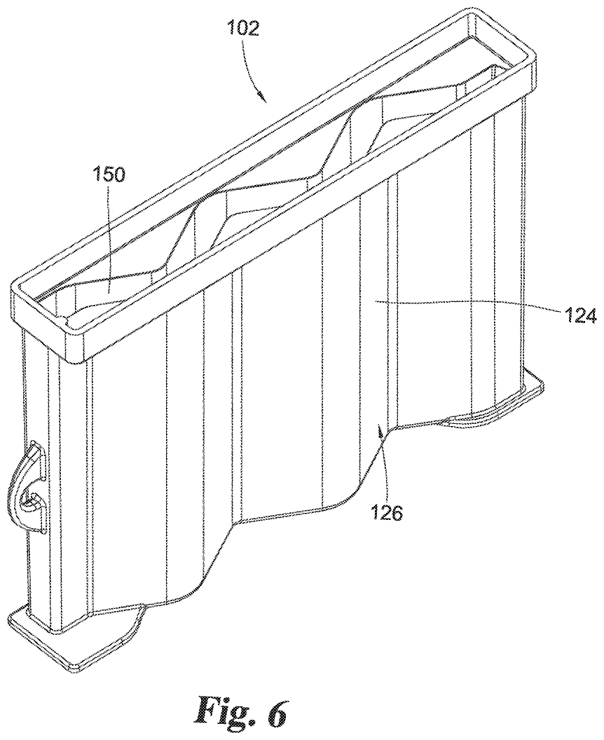

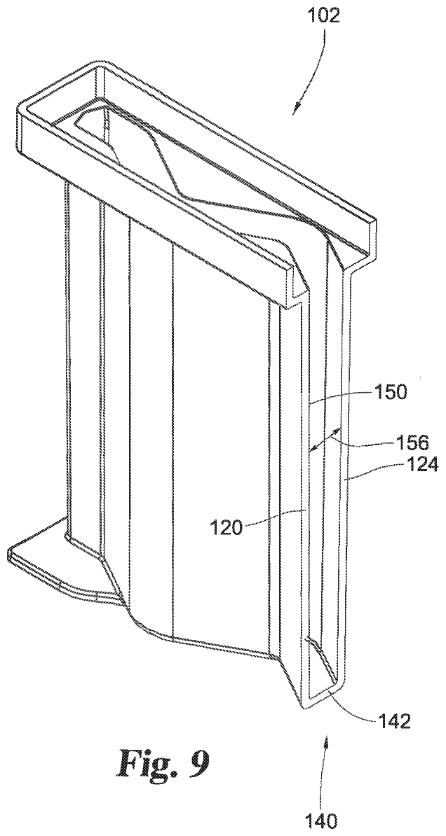

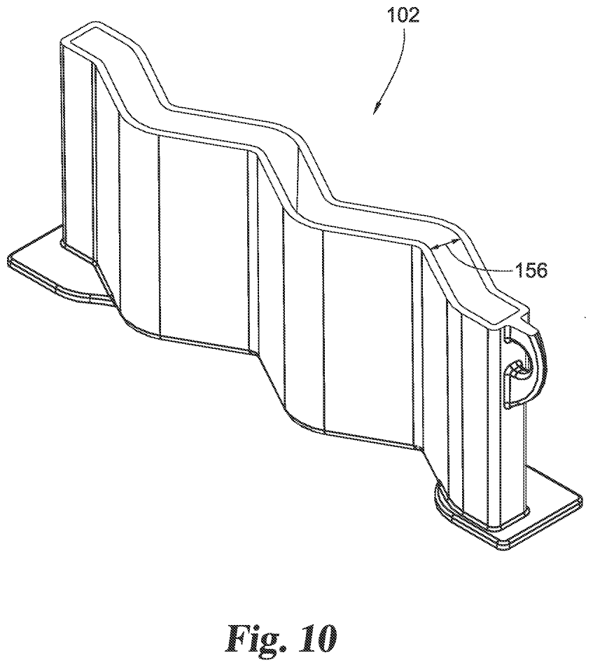

FIG. 1 illustrates a cryostorage container 100 including a body 102. FIG. 2 is a perspective view of the body 102. FIGS. 6, 7, and 8 are perspective views of the body 102 of the cryostorage container 100. FIGS. 9 and 10 are cross-sectional views of the body 102 of the cryostorage container 100. Referring to FIGS. 1, 2, and 6-10, the body 102 defines a chamber 110 for holding a volume of liquid. The chamber 110 is defined by one or more walls of the body 102. For example, the body 102 can have a first wall 120 extending along a first side 122 of the chamber and a second wall 124 extending along a second side 126 of the chamber 110 that opposes the first side 122. The chamber 110 can also be defined by a first end wall 130 positioned at a first end 132 of the chamber 110 and a second end wall 134 positioned at a second end 136 of the chamber 110. Extending along and closing the bottom 140 of the chamber 110 is a bottom wall 142. The walls can be rigid to facilitate retention of shape.

The first wall 120, the second wall 124, and the chamber 110 follow a serpentine path along a length L of the body 102. Each of the first wall 120 and the second wall 124 include a plurality of wall portions positioned at differing angles with respect to each other, or at alternating angles moving from the first end 132 to the second end 136. Along the serpentine path, the first wall 120 and the second wall 124 have a uniform characteristic dimension therebetween, which is a uniform distance 156 therebetween, as measured along a direction perpendicular to an inside surface 150 of the first wall 120. The uniform distance 156 is uniform across a planar cross-section parallel with the distance D. For example, the distance 156 between the second wall 124 and the first wall 120, as measured along a vector normal to the inside surface 150 of the first wall 120, can be at or between 5 mm and 7 mm. The first wall 120 can be parallel to the second wall 124 along the serpentine path.

The first wall 120 and the second wall 124 can also have the uniform distance 156 between them along the height of the body. Accordingly, the first wall 120 and the second wall 124 can be separated by the uniform distance 156 across the length L and/or the height H. Advantageously, having the uniform characteristic dimension between the first wall 120 and the second wall 124 along the length L and/or the height H of the body 102, wherein the value of the uniform characteristic dimension is relatively small, aids in maintaining a substantially uniform freeze profile along the length L and/or the height H of the body 102.

In practicality, an exact uniformity of the uniform characteristic dimension is difficult to manufacture, so some tolerance is acceptable. Further, in some embodiments, an indentation, recess, protrusion, or other nonuniform structural feature can be useful or necessary, for example, to facilitate manufacturing. These nonuniform structural features can affect the uniform characteristic dimension over a portion of a wall of the chamber 110. Accordingly, in some embodiments, the uniform characteristic dimension can range, e.g., within 0.01 mm, 0.1 mm, 0.5 mm, 1.0 mm, or 2.0 mm. In some embodiments, the uniform characteristic dimension can range, for example, to deviate 0.1%, 0.5%, 1%, 2%, 5%, 10%, 20%, or 25% from the uniform characteristic dimension's target value or median value. Generally, higher uniformity of the uniform characteristic dimension is desirable to increase uniform cooling and/or thawing rates of the contained biological samples/materials.

In some embodiment, the uniform characteristic dimension can be uniform, within any of the ranges of the above-described tolerances, for 90% or more, 95% or more, or 99% or more of the perpendicular distances between the first wall 120 and the second wall 124.

In some embodiments, the walls can slightly diverge from one another along a direction extending from a bottom 140 of the body 102 to a top 160 of the body 102. This divergence can facilitate releasing the body 102 from a mold during manufacturing while having no significant impact on varying the freeze profile along the height H of the body 102.

Advantageously, serpentine chambers having a width between inner walls, a height, and an overall length can provide a greater volume for liquid storage than linear chambers of the same width, height, and overall length. Accordingly, serpentine chambers arranged for a cryostorage box can store more liquid than a linear container of similar dimensions (that is, having equal overall lengths).

The thermal resistance of the first wall 120 and the second wall 124 is preferably uniform along the length L and height H. For example, the first wall 120 and/or the second wall 124 can have a uniform material thickness along the length L and the height H. The first wall 120 and the second wall 124 can have the same thermal resistance to promote uniform thermal transfer during freeze/thaw cycles.

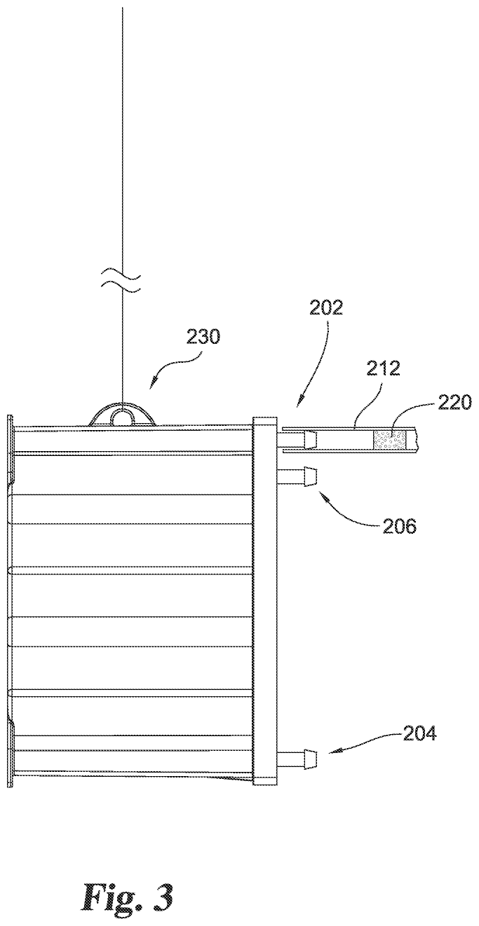

Referring additionally to FIG. 3, the cryostorage container includes one or more openings arranged to allow access to portions of the chamber 110. For example, a first opening 202 of the cryostorage container may communicate with the first end 132 of the chamber 110, a second opening 204 can communicate with the second end 136 of the chamber 110, and a third opening 206 can communicate with either the first end 132 or the second end 136 of the chamber 110.

Referring to FIGS. 1-3, one or more of the openings 202, 204, 206 can be associated with a connector 207 (e.g., a luer fitting or a barbed fitting) arranged for coupling to a liquid filling device 212 (e.g., a tube, a syringe, a supply bag, or a pump). For example, in the illustrated embodiment, the connectors 207 are barbed fittings configured to fit with resistance and/or interference within flexible tubing. The liquid filling device 212 is shown in FIG. 3 as flexible tubing connected to the barbed fitting of opening 202. The flexible tubing can be weldable (e.g., heat-sealable) such that inner surfaces of the flexible tubing can be welded together to close a lumen of the flexible tubing and seal the corresponding opening of the cryostorage container 100.

One or more of the openings 202, 204, 206 can be arranged for filling and/or draining liquid from the chamber 110. In some embodiments, one or more of the openings 202, 204, 206 can be high throughput fill ports. In some embodiments, at least one of openings 202, 204, 206 communicating with the chamber 110 can be arranged to vent air into and/or out of the chamber 110. For example, when liquid is being drained from the chamber 110, a vent opening arranged to vent air allows air to enter the chamber 110 and when liquid is being filled into the chamber 110, the vent opening allows air to exit the chamber 110. In the illustrated embodiments, the first opening 202 at the first end of the chamber 110 is arranged for venting, the second opening 204 at the second end of the chamber is arranged for draining, and the third opening 206 is arranged for filling the chamber with liquid.

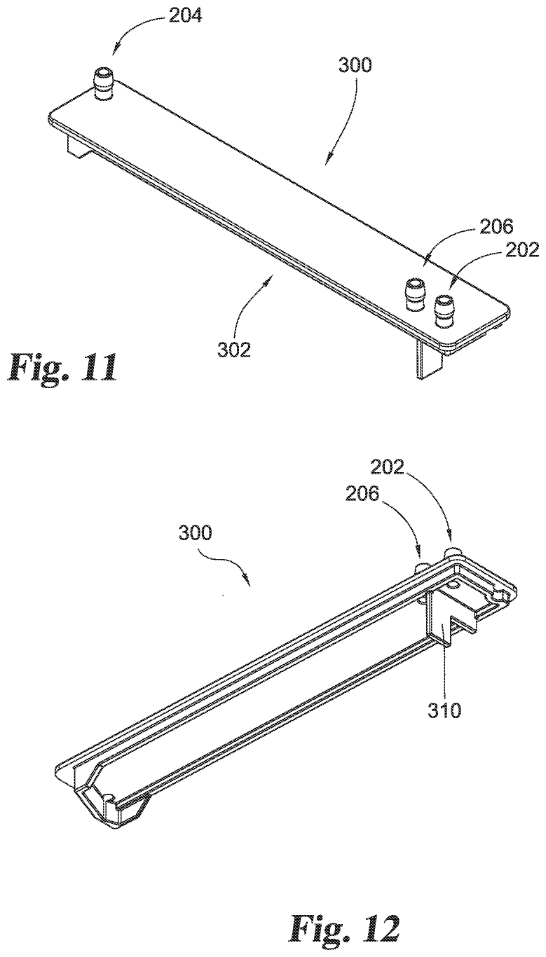

In some embodiments, the cryostorage container 100 can include a lid. FIGS. 11 and 12 are perspective views of a lid 300 of the cryostorage container 100. Referring to FIGS. 1, 2, 11, and 12, the lid 300 can close an end or side of the chamber 110. For example, in the illustrated embodiments, an upper portion of the chamber 110 defined by the top 160 of the body 102 is closed by the lid 300. The lid 300 can include one or more of the openings 202, 204, 206.

A bottom side 302 of the lid 300 may include a wall 310 extending between the vent opening (e.g., first opening 202) and the fill opening (e.g., third opening 206). The wall 310 prevents fluid entering through the fill opening from being immediately drawn out the vent opening, particularly when a negative pressure is applied to the vent opening to draw gas out the chamber 110.

Referring to FIG. 3, a filter element 220 can be associated with the vent opening and can be arranged to filter air entering the chamber 110 while the chamber 110 is being drained. The filter element 220 can be a micro-filter, more specifically a filter that provides a microbial barrier. Such a micro-filter may be, for example, a 3 μm, 0.2 μm, or 0.1 μm sterile porous micro-filter. In other embodiments, a micro-filter may have a different structure. The filter element 220 can be arranged to be gas permeable but is generally hydrophobic (i.e., resistant to passage of the liquid sample or cell suspension being stored within the container). Thus, the filter element 220 is capable of retaining the suspension within the cryostorage container 100 and venting gas out of the cryostorage container 100 when the cryostorage container 100 is being filled (either partially or completely) and venting gas into the cryostorage container 100 when suspension is being withdrawn from the cryostorage container 100. The filter element 220 is shown positioned/retained within the flexible tubing 212 associated with the first opening 202, though the filter element 220 can be arranged otherwise in the air path through the vent opening.

The cryostorage container can include a hanging element 230, which can be a loop, as shown in FIGS. 1-3, or can be another now-known or future-developed arrangement for suspending the cryostorage container above a surface. The hanging element 230 can be arranged such that when the cryostorage container 100 is suspended from the hanging element 230, as shown in FIG. 3, the drain opening (e.g, opening 204) is at the lowest point of the chamber 110. Accordingly, fluid will flow under force of gravity toward the drain opening. Such an arrangement can facilitate maximum or complete draining of fluid from the chamber 110, except for perhaps some residual fluid such as moisture or fluid film adhering to interior surfaces of the chamber 110. For example, the hanging element can be located on an opposing end from the drain opening and/or closer to a side opposing the side having the drain opening. The hanging element 230 can be off-center along an end of the cryostorage container 100.

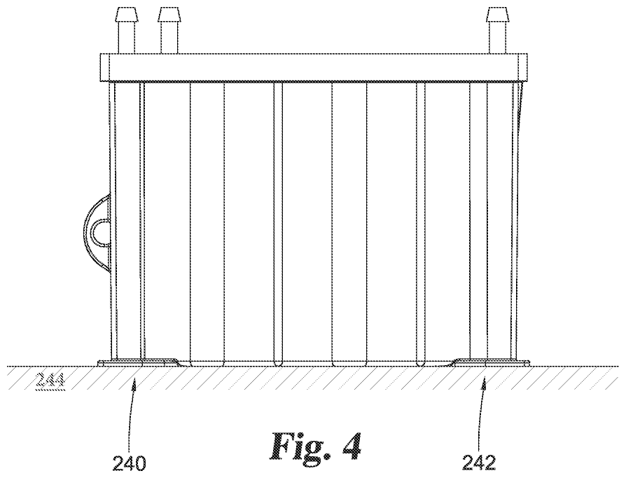

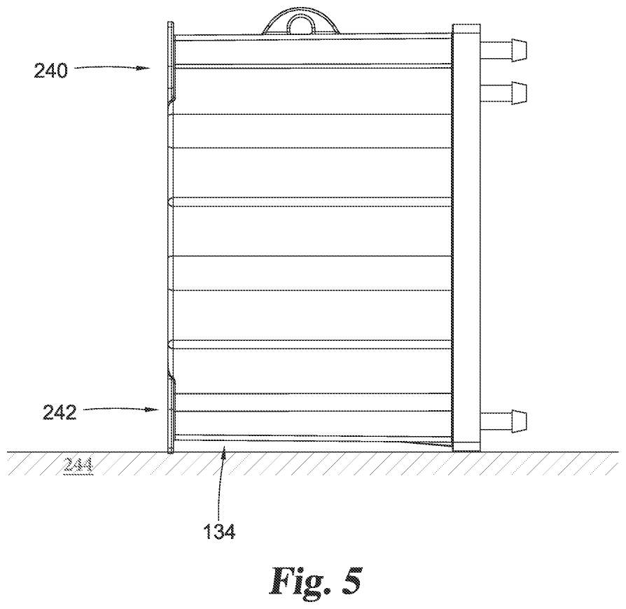

The cryostorage container 100 can be arranged to support itself on a level surface in various positions. For example, the cryostorage container 100 can be arranged to support itself in a filling configuration. FIG. 4 is a side view of the cryostorage container 100 in a filling configuration. In the filling configuration, the vent opening is elevated relative to at least 80% of the chamber. Accordingly, during filling, liquid rises toward the vent opening and gas within the chamber is expelled through the vent opening. The cryostorage container 100 can also be arranged to support itself in a draining configuration (see FIGS. 3 and 5), which can be the same or different from the filling configuration. In the draining configuration, the drain opening is positioned lower than at least 80% of the chamber.

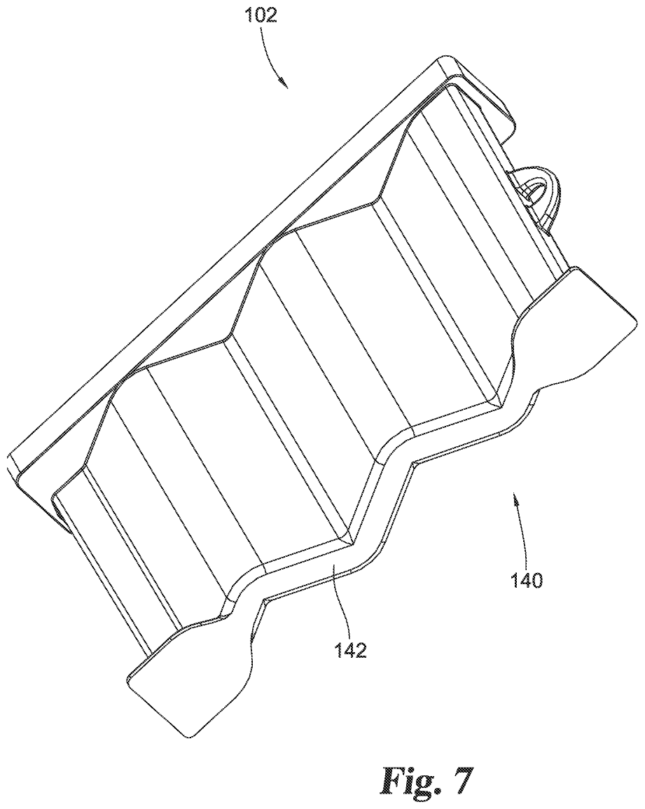

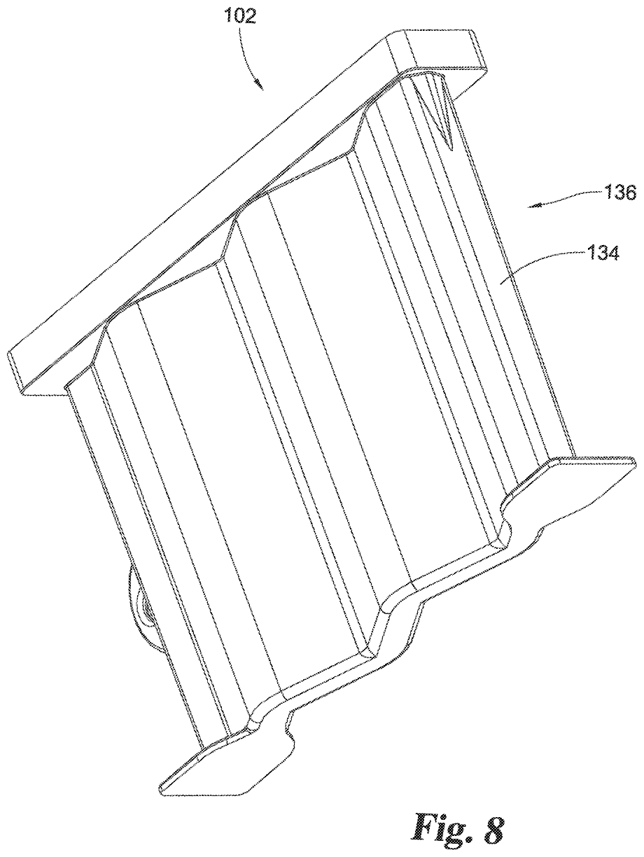

To support itself in one or more configurations (e.g., filling and/or draining configurations), the cryostorage container 100 can include one or more stands. For example, referring to FIGS. 2, 4, and 5, the illustrated embodiment of the cryostorage container 110 has a first stand 240 at the first end 132 and a second stand 242 at the second end 136. In the filling configuration, shown in FIG. 4, both the first stand 240 and second stand 242 are in contact with a supporting surface 244, and the vent opening and the drain/fill opening are elevated relative to the chamber 110. In the drain configuration, shown in FIG. 5, the cryostorage container 100 is supported on the supporting surface 244 by the second stand 242 and the drain opening is positioned below the chamber. The inside surfaces of the lowest wall(s) defining the chamber 110 (e.g., second end wall 134 shown in FIGS. 5 and 8) slope downwardly towards the drain opening in the drain configuration and/or when the cryostorage container 100 is supported by the hanging element 230 so that substantially all the fluid, except perhaps residual fluid (e.g., <1% by volume), can be removed from the chamber 110.

In some embodiments, the cryostorage container 100 can have a height of about 80 mm, a length of about 125 mm, a width of about 20 mm, and a volume of about 48 ml. In some embodiments, the cryostorage container 100 has a volume in a range of 100 ml to 250 ml.

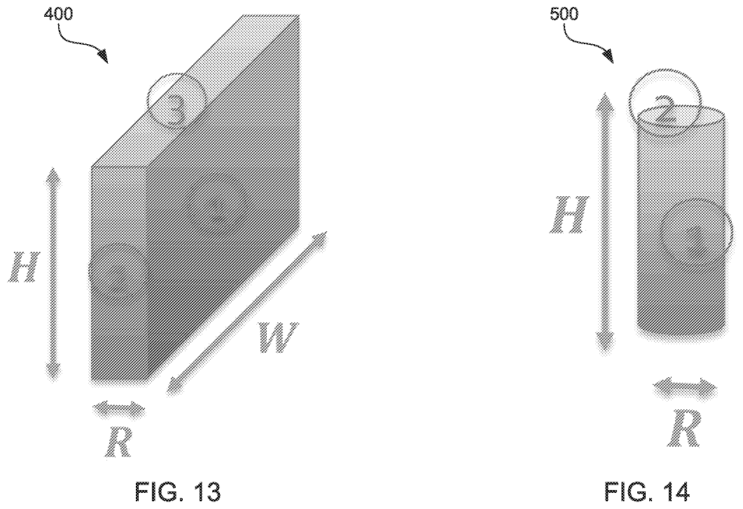

As described briefly above, cryopreservation containers with relatively large capacities according to the present invention are provided with uniform characteristic dimensions and high dimensional aspect ratios to facilitate consistency and uniformity of freeze/thaw rates. Such containers can have serpentine chambers, such as the cryostorage container 100 described above. Alternatively, such containers can have other shapes. Several exemplary shapes of chambers of such containers are illustrated in FIGS. 13-26 and 32-34.

Referring now to FIG. 13, a cryostorage chamber 400 having a three-dimensional rectangular shape is illustrated. The chamber 400 has a uniform characteristic dimension R, more specifically a thickness dimension. The chamber 400 also has a height dimension H and a width dimension W. Aspect ratios for the chamber 400 are defined as follows:

$${A}_{r 2} = \frac{H}{R}$$

$${A}_{r 3} = \frac{W}{R}$$

Surface areas of the chamber 400 relate to the aspect ratios as follows:

$$\frac{S {A}_{1}}{S {A}_{2}} = \frac{2 H W}{2 H R} = \frac{W}{R} = {A}_{r 3}$$

$$\frac{S {A}_{1}}{S {A}_{3}} = \frac{2 H W}{2 H R} = \frac{H}{R} = {A}_{r 2}$$

For relatively high aspect ratios, or relatively high ratios of SA1 to SA2 or SA1 to SA3, the characteristic dimension R significantly affects, and provides substantial consistency of, the freeze profile of the chamber 400. Such relatively high aspect ratios may be, for example, between 1 to 1 and 2 to 1, preferably between 2 to 1 and 5 to 1, and more preferably between to 1 and 20 to 1.

Referring to FIG. 14, a cryostorage chamber 500 having a cylindrical shape is illustrated. The chamber 500 has a uniform characteristic dimension R, more specifically a diameter. The chamber 500 also has a height dimension H. The aspect ratio for the chamber 500 is defined as follows:

$${A}_{r} = \frac{H}{R}$$

Surface areas of the chamber 500 relate to the aspect ratios as follows:

$$\frac{S {A}_{1}}{S {A}_{2}} = \frac{π R H}{( π {R}^{2} ) / 2} = \frac{2 H}{R} = 2 {A}_{r}$$

For a relatively high aspect ratio, or a relatively high ratios of SA1 to SA2, the characteristic dimension R significantly affects, and provides substantial consistency of, the freeze profile of the chamber 500. Such a relatively high aspect ratio may be, for example, between 1 to 1 and 2 to 1, preferably between 2 to 1 and 5 to 1, and more preferably between to 1 and 20 to 1.

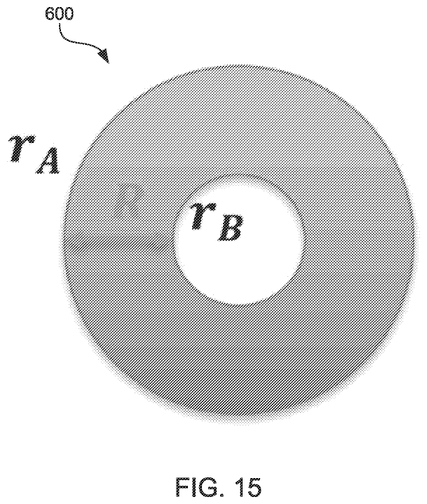

Referring to FIG. 15, a cryostorage chamber 600 having a three-dimensional hollow cylindrical shape, or donut shape, is illustrated. The chamber 600 has a uniform characteristic dimension R, more specifically a difference between an outer radius TA and an inner radius ra.

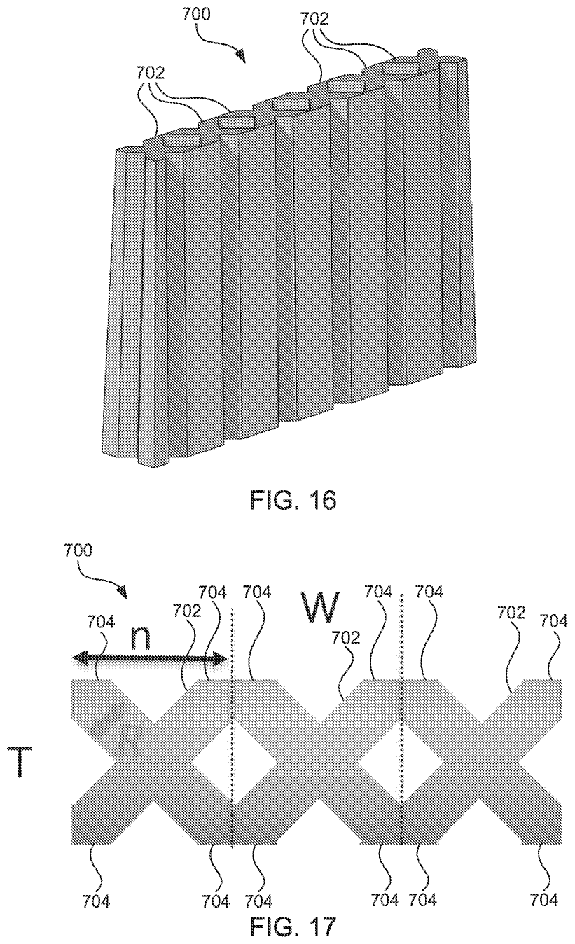

Referring to FIGS. 16 and 17, a cryostorage chamber 700 having a plurality of conjoined three-dimensional square X shapes 702 is illustrated. Referring specifically to FIG. 17, the chamber 700 has a uniform characteristic dimension R, more specifically a perpendicular width of each leg of the X shapes 702. The chamber 700 also includes an overall thickness T, an overall width W, and a number n of the X shapes 702. Because the X shapes 702 are square, W/n=T. The legs of the X shapes 702 have pointed ends 704. In some embodiments, an optimal value of R/T provides the chamber 700 with a relatively large perimeter without overly reducing the area of the chamber 700. In some embodiments, the value of R/T is in a range of 0.4 to 0.5, more specifically about 0.45. In some embodiments, the chamber 700 has a height of about 80 mm, an overall width W of about 120 mm, an overall thickness T of about 20 mm, and a volume of about 103 ml.

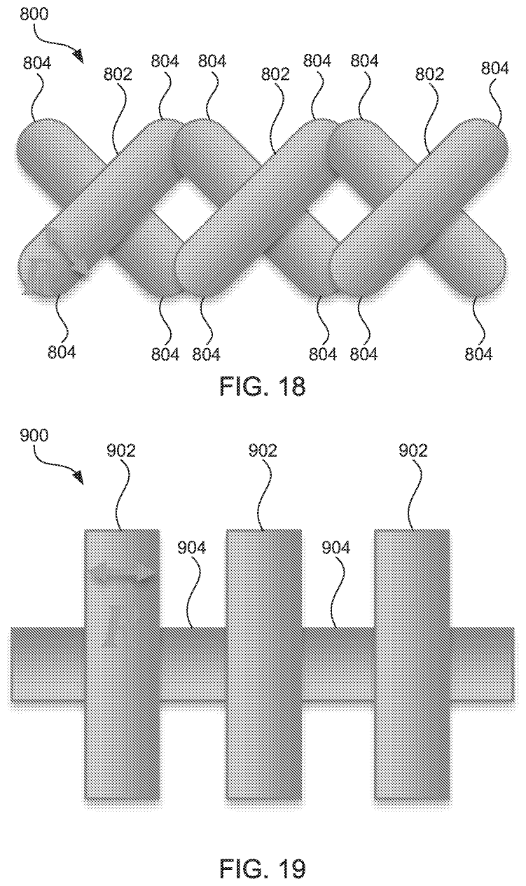

Referring to FIG. 18, a cryostorage chamber 800 having a plurality of conjoined three-dimensional square X shapes 802 is illustrated. The chamber 800 is the same as the chamber 700 described above, except that the legs of the X shapes 802 have rounded ends 804.

Referring to FIG. 19, a cryostorage chamber 900 having a plurality of three-dimensional rectangular shapes 902 is illustrated. Each shape 902 includes a uniform characteristic dimension R, more specifically a thickness dimension. The shapes 902 are connected by a plurality of connectors 904.

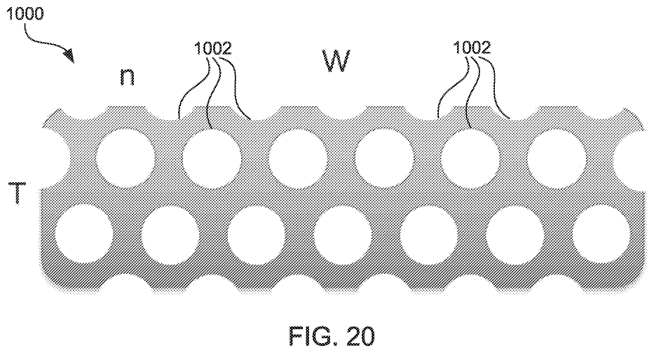

Referring to FIG. 20, a cryostorage chamber 1000 having a three-dimensional rectangular shape with a plurality of cylindrical voids 1002 is illustrated. The chamber 1000 includes an overall thickness T, an overall width W, and a number n of segments. The chamber 1000 has a uniform characteristic dimension (not labeled), more specifically a perpendicular width between the cylindrical voids 1002.

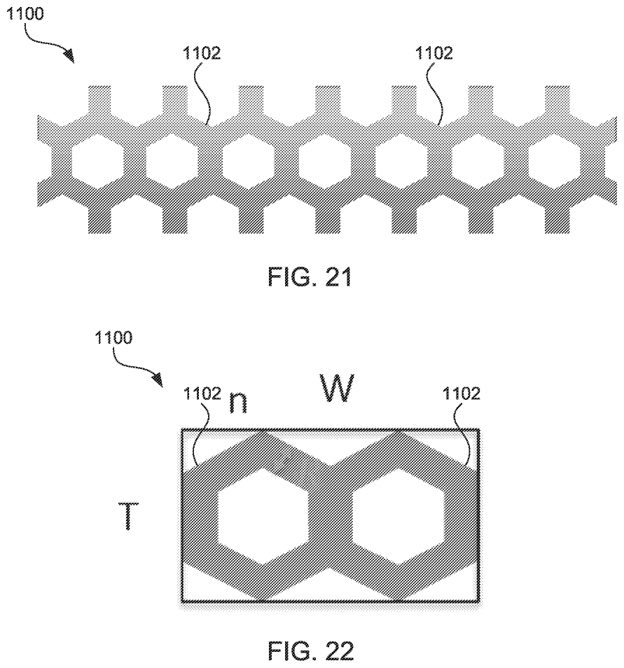

Referring to FIGS. 21 and 22, a cryostorage chamber 1100 having a plurality of conjoined three-dimensional hollow hexagon shapes 1102 is illustrated. Referring specifically to FIG. 22, the chamber 1100 has a uniform characteristic dimension R, more specifically a perpendicular width of each of the hollow hexagon shapes 1102. The chamber 1100 also includes an overall thickness T, an overall width W, and a number n of the hollow hexagon shapes 1102.

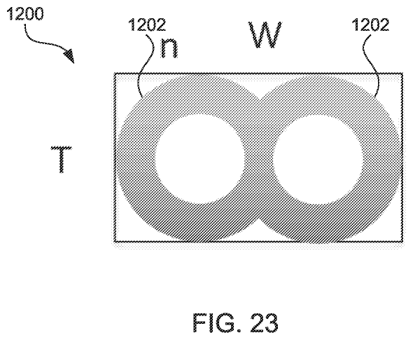

Referring to FIG. 23, a cryostorage chamber 1200 having a plurality of three-dimensional hollow cylinders 1202 is illustrated. The chamber 1200 includes an overall thickness T, an overall width W, and a number n of the hollow cylinders 1202.

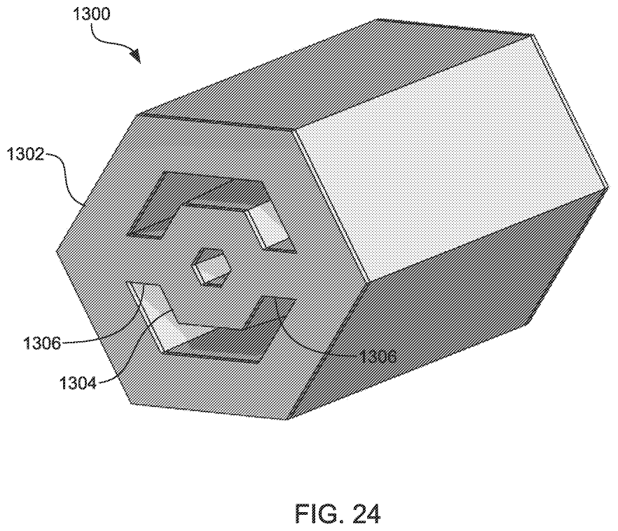

Referring to FIG. 24, a cryostorage chamber 1300 having nested hollow hexagon shapes is illustrated. More specifically, the chamber 1300 includes an outer hollow hexagon shape 1302, an inner hollow hexagon shape 1304, and connectors 1306 joining the hollow hexagon shapes 1302, 1304. The chamber 1200 may have a longitudinal height of about 100 mm, a width of about 60 mm, a thickness of about 51 mm, and a volume of about 108 ml.

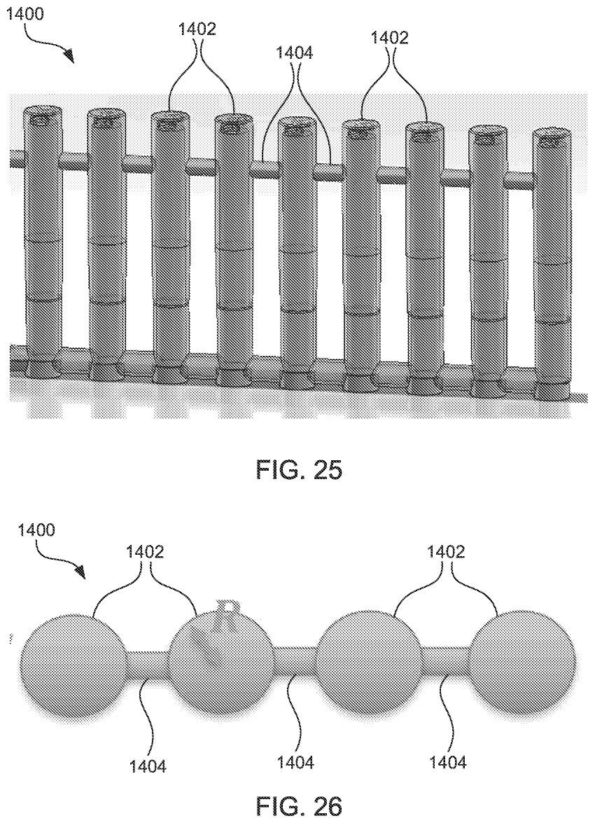

Referring to FIGS. 25 and 26, a cryostorage chamber 1400 having a plurality of cylinders 1402 is illustrated. Referring specifically to FIG. 26, Each cylinder 1402 includes a uniform characteristic dimension R, more specifically a radius. The cylinders 1402 are connected by a plurality of connectors 1404.

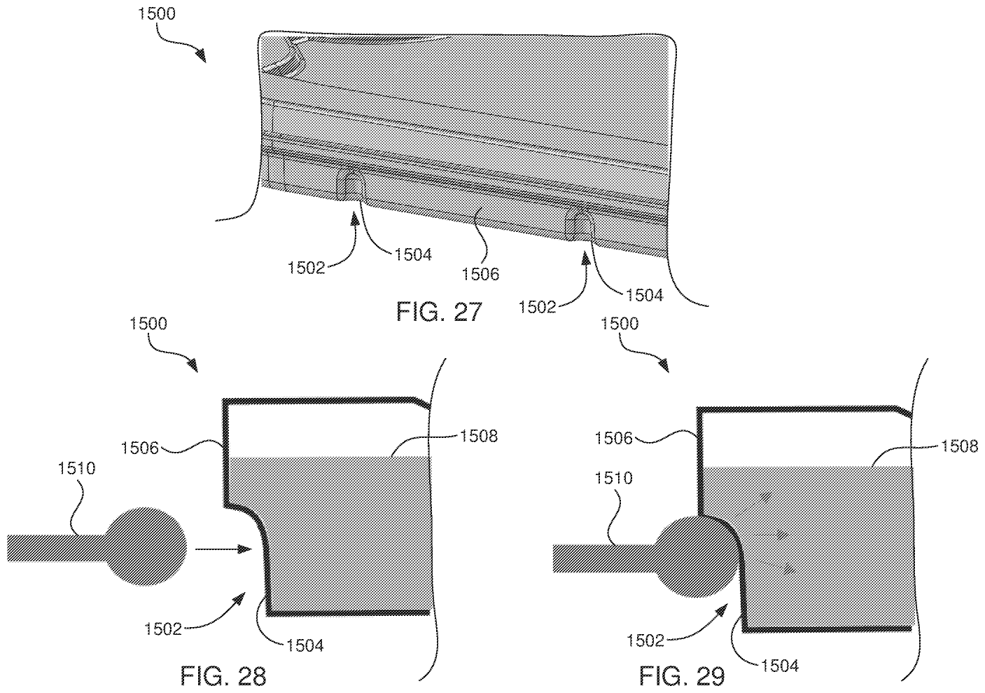

Referring to FIGS. 27-29, a cryostorage container 1500 is illustrated, which may generally have the same structure and dimensions as any of the cryostorage containers described herein. Additionally, the cryostorage container 1500 includes one or more nucleation sites 1502 (illustratively, two nucleation sites) that facilitate ice crystal formation. Illustratively, each nucleation site 1502 is formed as an indentation or recess 1504 in a wall 1506 of the cryostorage container 1500. In some embodiments, a method of freezing a sample facilitated by the nucleation sites 1502 is as follows. First, a sample 1508 is delivered to the cryostorage container 1500. The sample 1508 is then cooled (for example, via placement in a controlled rate freezer) to freezing or below freezing (that is, a supercool state where water remains liquid but is very close to freezing via random ice nucleation). Next and as shown specifically in FIG. 28, a nucleation trigger 1510 (for example, a metallic object cooled below freezing) is moved toward one of the nucleation sites 1502. As shown specifically in FIG. 29, the nucleation trigger 1510 then contacts the nucleation site 1502. The nucleation trigger 1510 thereby causes ice nucleation at the nucleation site 1502, and ice nucleation then propagates outwardly from the nucleation site 1502.

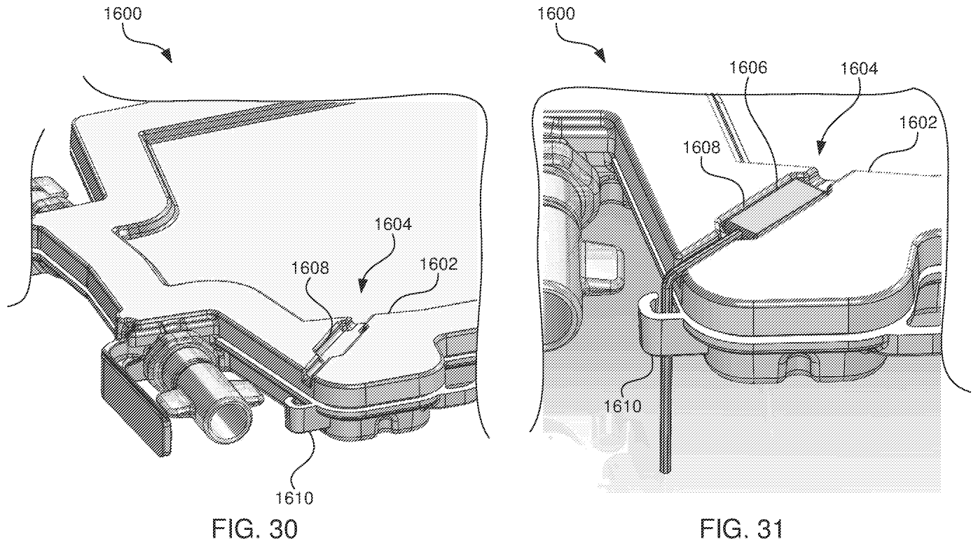

Referring to FIGS. 30 and 31, a cryostorage container 1600 is illustrated, which may generally have the same structure and dimensions as any of the cryostorage containers described herein. Additionally, one or more walls 1602 of the container 1600 may include one or more mounting features 1604 that each receive a thermocouple 1606 (FIG. 31). The mounting feature 1604 inhibits detachment of the thermocouple 1606 from the container 1600, which could lead to temperature deviation in manufacturing records. In addition, the mounting feature 1604 facilitates consistent location of temperature measurements across samples and batches. Illustratively, the mounting feature 1604 is formed as an external recess or channel 1608 in the wall 1602 and a wire retention element 1610.

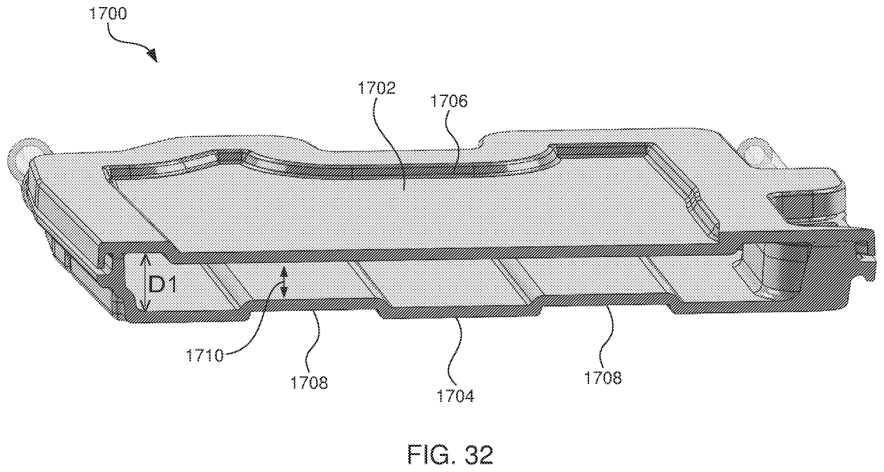

Referring to FIG. 32, a cryostorage container 1700 is illustrated, which may generally have the same structure and dimensions as any of the cryostorage containers described herein. The container 1700 includes opposing upper and lower walls 1702, 1704 that define a chamber 1705. A uniform characteristic dimension D1, in this instance, can vary, as discussed above, to include one or more recesses for particular purposes other than promoting consistent and uniform freeze/thaw rates. In this instance, the cryostorage container 1700 facilitates visual inspection, optical quantitative measurement, and other types of non-destructive testing of the contents of the container 1700. The opposing upper and lower walls 1702, 1704 can be transparent or include transparent portions that facilitate microscopy and spectroscopy. Such processes in turn facilitate in-process measurements, diagnostics, and early identification of sample issues. In some embodiments, the upper wall 1702 and/or the lower wall 1704 include the one more recesses to inhibit marring of the respective walls 1702, 1704 during manufacturing, distribution, and/or use.

Illustratively, the upper wall 1702 includes one recess 1706 and the lower wall 1704 includes two recesses 1708. In some embodiments, the upper wall 1702 and the lower wall 1704 define an optical path length 1710 therebetween, and the optical path length 1710 may be uniform across a set of containers of various volumes. The one recess 1706 and the two recesses 1708 can be deep enough to fulfill their intended purpose of reducing or preventing damage to the walls 1702, 1704 while also being shallow enough to maintain sufficient uniformity of the uniform characteristic dimension D1 (e.g., within the tolerances discussed above) and sufficient consistency/uniformity of the freeze/thaw rates in the chamber.

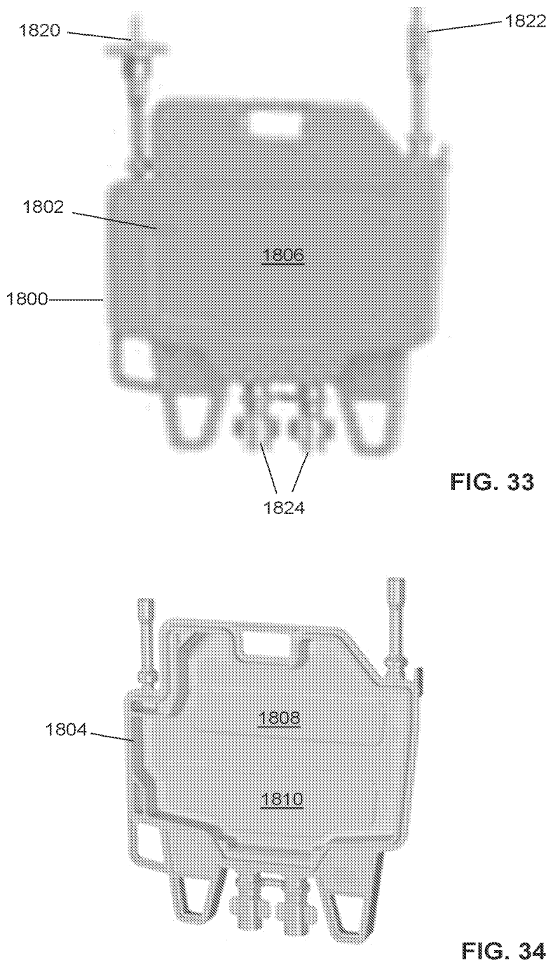

FIG. 33 shows an external side of an upper wall 1802 of a cryostorage container 1800 and FIG. 34 shows an internal side of a lower wall 1804 of the cryostorage container 1800. FIGS. 33 and 34 also show vent lines 1820, fill lines 1822, and spike ports 1824. Similar to the embodiment of FIG. 32, a uniform characteristic dimension (not shown but comparable to D1 of FIG. 33), in this instance, can vary at particular locations or regions between the upper wall 1802 and the lower wall 1804 due to the inclusion of one or more recesses for particular purposes other than promoting consistent and uniform freeze/thaw rates. The upper wall 1802 includes a first recess 1806 and the lower wall 1804 includes a second recess 1808 and a third recess 1810. The first recess 1806, the second recess 1808, and the third recess 1810 can be deep enough to fulfill their intended purpose while also being shallow enough to maintain sufficient uniformity of the uniform characteristic dimension (e.g., within the tolerances discussed above) and sufficient consistency/uniformity of the freeze/thaw rates in the chamber. For example, the uniform characteristic dimension, or perpendicular distance between the upper wall 1802 and the lower wall 1804 can be 10 mm at its largest where there are no recesses and 8 mm at its smallest where there is a recess in each wall 1802, 1804. Accordingly, it could be considered that the uniform characteristic dimension is 10 mm, with a deviation of minus 20% where the perpendicular distance between the upper wall 1802 and the lower wall 1804 extends between two recesses. In this example, a maximum height H of the cryostorage container 1800 is about 100 mm, such that an aspect ratio is on the order of 10 to 1.

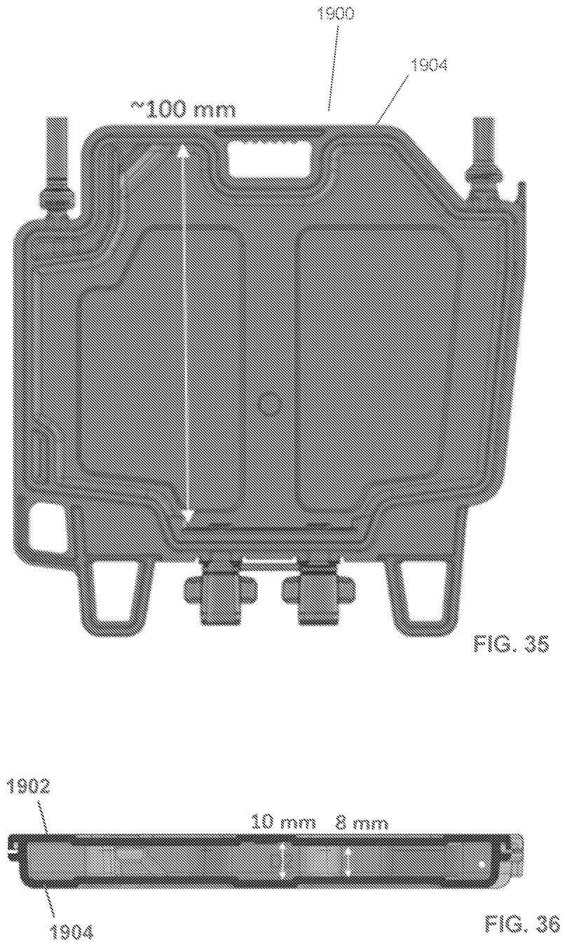

FIG. 35 shows an internal side of a lower wall 1904 of a cryostorage container 1900, and FIG. 36 shows a planar cross section of the cryostorage container 1900 parallel to a uniform characteristic dimension D2. The cryostorage container 1900 is like the cryostorage chamber 1800, but with an upper wall 1902 having two recesses, and with different shapes of the recesses.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A cryostorage container, comprising:
a first body defining a chamber, the first body including a plurality of walls, the plurality of walls including:
a first wall extending along a first side of the chamber; and
a second wall extending along a second side of the chamber, the second side opposite the first side relative to the chamber,
the chamber having a volume in a range of 25 mL to 250 mL, the chamber having a uniform characteristic dimension extending perpendicular to the first wall and the second wall from the first wall to the second wall, the uniform characteristic dimension being uniform across a planar cross-section of the chamber parallel to the uniform characteristic dimension.

2. The cryostorage container of claim 1, wherein the uniform characteristic dimension is uniform for at least 90% of all distances extending from the first wall to the second wall and being perpendicular to the first wall and the second wall.

3. The cryostorage container of claim 1, further comprising:
a vent opening in fluid communication with the chamber; and
a drain opening in fluid communication with the chamber.

4. The cryostorage container of claim 3, further comprising a filter associated with the vent opening to filter air entering and/or exiting the chamber.

5. The cryostorage container of claim 3, further comprising heat-sealable tubing attached to at least one from the group consisting of the drain opening and the vent opening.

6. The cryostorage container of claim 3, wherein the vent opening is positioned at a first end of the chamber and the drain opening is positioned at a second end of the chamber.

7. The cryostorage container of claim 1, wherein the chamber follows a serpentine path.

8. The cryostorage container of claim 7, wherein the body further includes:
a first end wall at a first end of the chamber, the first end wall connecting the first wall and the second wall; and
a second end wall at a second end of the chamber, the second end wall opposite the first end wall relative to the chamber,
wherein the serpentine path extends along a length between the first end wall and the second end wall.

9. The cryostorage container of claim 7, wherein the first wall includes a plurality of wall portions positioned at different angles with respect to each other, wherein the second wall includes a plurality of wall portions positioned at different angles with respect to each other, each wall portion of the first wall approximately parallel with and opposite one of the wall portions of the second wall.

10. The cryostorage container of claim 7, wherein the serpentine path is defined at least partially by a series of curves bending in alternating directions, the series of curves separated by straight wall portions.

11. The cryostorage container of claim 7, wherein the serpentine path is defined by a series of straight wall portions arranged in a chevron pattern.

12. The cryostorage container of claim 1, wherein the uniform characteristic dimension is at or between 5 mm and 10 mm.

13. The cryostorage container of claim 1, further comprising a hanger opening arranged to receive an IV bag hanger.

14. The cryostorage container of claim 13, wherein the hanger opening is positioned at an opposite end of the cryostorage container from the drain opening such that when the container hangs freely from the IV bag hanger, the drain opening is at a lowest portion of the serpentine chamber.

15. The cryostorage container of claim 14, wherein the hanger opening is off-center along the end of the cryostorage container.

16. The cryostorage container of claim 1, further comprising a nucleation site facilitating ice crystal formation.

17. The cryostorage container of claim 16, wherein the nucleation site comprises an indentation formed in the plurality of walls.

18. The cryostorage container of claim 1, wherein the plurality of walls comprise a mounting feature, and wherein the cryostorage container further comprises a thermocouple carried by the mounting feature.

19. The cryostorage container of claim 18, wherein the mounting feature comprises a recess.

20. The cryostorage container of claim 19, wherein the mounting feature further comprises a wire retention element.

21. The cryostorage container of claim 1, wherein the plurality of walls includes transparent wall portions.

22. The cryostorage container of claim 21, wherein the transparent wall portions are recessed.

23. The cryostorage container of claim 1, further comprising:
a second body defining a second chamber, the second body including a second plurality of walls, the second plurality of walls including:
a third wall extending along a first side of the second chamber; and
a fourth wall extending along a second side of the second chamber, the second side of the second chamber opposite the first side of the second chamber relative to the second chamber,
the second chamber having a second volume in a range of 25 mL to 250 mL, the second chamber having a second uniform characteristic dimension extending perpendicular to the third wall and the fourth wall from the third wall to the fourth wall, the second uniform characteristic dimension being uniform across a planar cross-section of the second chamber parallel to the second uniform characteristic dimension,
the first chamber in fluid communication with the second chamber.

24. The cryostorage container of claim 23, further comprising:
a third body defining a third chamber, the third body including a third plurality of walls, the third plurality of walls including:
a fifth wall extending along a first side of the third chamber; and
a sixth wall extending along a second side of the third chamber, the second side of the third chamber opposite the first side of the third chamber relative to the third chamber,
the third chamber having a third volume, the third chamber having a third uniform characteristic dimension extending perpendicular to the fifth wall and the sixth wall from the fifth wall to the sixth wall, the third uniform characteristic dimension being uniform across a planar cross-section of the third chamber parallel to the third uniform characteristic dimension,
the second chamber in fluid communication with the third chamber

25. A cryostorage container comprising:
a chamber having a volume; and
wall portions defining the chamber, the wall portions each having an interior surface facing the chamber and an exterior surface opposing the interior surface,
each interior surface having a surface area, a ratio of a sum of the surface areas of the interior surfaces to the volume of the chamber being at or between 1 to 5 and 2 to 5.

26. The cryostorage container of claim 25, wherein the wall portions defining the chamber follow a serpentine path from a first end of the chamber to a second end of the chamber.

27. The cryostorage container of claim 26, wherein the chamber has a length extending from the first end to the second end and a uniform width along the length.

28. The cryostorage container of claim 27, wherein the width is at or between 5 mm and 10 mm.

29. The cryostorage container of claim 25, wherein the wall portions have a thermal conductivity at or between 0.16×10⁻³ watts per Kelvin and 0.32×10⁻³ watts per Kelvin at room temperature.

30. The cryostorage container of claim 25, wherein the wall portions comprise a material having a thermal conductivity at or between 0.10 to 0.20 W/m K at room temperature.
